(19) United States
(12) Patent Application Publication
(10) Pub. No.: US 2026/0243760 A1
(43) Pub. Date: Aug. 20, 2026

(54) METHOD AND SYSTEM FOR OPTICALLY BASED IMMUNOASSAYS

(71) Applicants: Martin Huber, Menlo Park, CA (US); Ossama Assad, San Carlos, CA (US)

(72) Inventors: Martin Huber, Menlo Park, CA (US); Ossama Assad, San Carlos, CA (US)

(21) Appl. No.: 19/165,225

(22) PCT Filed: Mar. 20, 2024
(86) PCT No.: PCT/US2024/020630

§ 371 Date: Sep. 15, 2025

Related U.S. Application Data
(60) Provisional application No. 63/456,554, filed on Apr. 3, 2023.

(51) Int. Cl. G01N 33/543; G01N 21/64

(52) U.S. Cl. CPC G01N 33/54326; G01N 21/6428; G01N 21/648; G01N 33/54386; G01N 2021/6439; G01N 2470/04

(57) ABSTRACT

The invention is directed to methods and systems for optically based immunoassays for one or more analytes, such as proteins, employing subwavelength hole arrays.

### BACKGROUND

Sensitive, selective and quantitative assaying of biomolecules or other analytes in easily obtainable biological fluids such as serum and urine are key to improved patient outcomes, especially in such fields as cancer therapy. To this end, a wide variety of immunoassays employing particles, such as superparamagnetic beads, have been developed, e.g. Duffy, LabChip, 23, 818 (2023); Tekin et al, LabChip, 13: 4711(2013 ); Tang et al, The Analyst, 138: 981 (2013); Xue et al, Nature Reviews Materials, 5: 931-951 (2020); Farka et al, Chem. Rev., 117: 9973-10042 (2017); and the like. Many of these assays employ electrical signals for detection, such as, current, voltage or resistive changes across a nanopore. Such approaches are cost effective for single nanopore measurements, but are more expensive and complex if high throughput parallel measurements are desired, such as when a diagnostic value or score depends on the levels of multiple analytes present in very low concentrations, such as cancer biomarker proteins in serum, e.g. Fried et al, Nano Letters, 22: 869-880 (2022); Landegren et al, Anal. Chem., 84: 1824-1830 (2012).

In view of the above, the availability of new methods and compositions for low cost immunoassays based on parallel optical detection would advance a host of fields where sensitive monitoring of multiple analytes is required, such as in environmental sampling, drug testing, allergy testing, cancer diagnostics, and the like.

### SUMMARY OF THE INVENTION

The present invention is directed to assays, particularly immunoassays, for detecting and/or quantifying one or more analytes, such as proteins, in a sample. In some embodiments, optically labeled binding compounds specific for selected analytes are combined with a sample to form one or more capture complexes. The presence and/or quantities of the analytes in the sample are determined by evaluating the optical signals generated by the capture complexes as they pass through holes of a subwavelength hole array being illuminated by an excitation beam.

The invention includes methods for determining the presence or absence and/or amounts of one or more analytes in a sample. In some embodiments, methods of the invention comprise: (a) combining a sample with capture agents and associated detection agents to form one or more capture complexes each having one or more optical labels, wherein each capture agent and associated detection agents is specific for an analyte; (b) translocating capture complexes through a hole array illuminated with excitation light, wherein the hole array comprises a solid phase membrane and an opaque layer co-extensive therewith and a plurality of holes there through, such that (i) the opaque layer substantially prevents excitation light from passing through the hole array, and (ii) each hole has an exit and a diameter less than a wavelength of excitation light such that a signal generation region is formed adjacent to its exit; (c) detecting for each hole the one or more optical signals of the translocated capture complexes; and (d) determining the presence or absence and the quantities of each of the one or more analytes in the sample from the optical signals collected from the capture complexes.

In other embodiments, methods of the invention comprise (a) providing a hole array comprising a solid phase membrane and an opaque layer co-extensive therewith and a plurality of holes there through, wherein (i) the opaque layer substantially prevents excitation light from passing through the hole array, and (ii) each hole has an exit and a diameter less than a wavelength of excitation light such that a signal generation region is formed adjacent to its exit whenever excitation light is incident to the opaque layer; (b) combining a sample with capture agents and associated detection agents to form one or more capture complexes each having one or more optical labels, wherein each capture agent and associated detection agents is specific for an analyte; (c) directing excitation light to the opaque layer of the hole array to produce signal generation regions at the exits of the holes; (d) translocating capture complexes through the hole array from a side opposite the opaque layer so that the one or more optical labels of each capture complex produce one or more optical signals as the capture complex passes through a signal generation region at the exits of the holes; (e) detecting for each hole the one or more optical signals of the translocated capture complexes; and (f) determining the presence or absence and the quantities of each of the one or more analytes in the sample from the optical signals collected from the capture complexes.

In methods of the invention for determining the presence and amounts of one or more analytes in a sample, one of ordinary skill in the art would understand that the absence of an analyte in a sample may also be determined (within the limit of assay sensitivity), for example, by including internal standards of known concentrations with a sample.

### BRIEF DESCRIPTIONS OF THE DRAWINGS

FIGS. 1A-1B illustrate diagrammatically an embodiment of a sandwich immunoassay comprising one capture antibody and one detection antibody.

FIGS. 1C-1D illustrate diagrammatically an embodiment of a competitive immunoassay in accordance with the invention.

FIG. 1E illustrates diagrammatically an embodiment for allergen testing in accordance with the invention.

FIGS. 1F-1G illustrate diagrammatically an embodiment of a sandwich immunoassay comprising one capture antibody and two detection antibodies.

FIG. 2 illustrates an optical system for use with embodiments of the invention.

FIG. 3 illustrates a microfluidic cartridge for delivering processed samples to a hole array.

### DETAILED DESCRIPTION OF THE INVENTION

The practice of the present invention may employ, unless otherwise indicated, conventional techniques and descriptions of organic chemistry, molecular biology (including recombinant techniques), biochemistry, micro-and nanostructure fabrication, and the like, which are within the skill of the art. Guidance for aspects of the invention is found in many available references and treatises well known to those with ordinary skill in the art, including, for example, Cao, Nanostructures & Nanomaterials (Imperial College Press, 2004); Levinson, Principles of Lithography, Second Edition (SPIE Press, 2005); Doering and Nishi, Editors, Handbook of Semiconductor Manufacturing Technology, Second Edition (CRC Press, 2007); Sawyer et al, Electrochemistry for Chemists, 2nd edition (Wiley Interscience, 1995); Bard and Faulkner, Electrochemical Methods: Fundamentals and Applications, 2nd edition (Wiley, 2000); Lakowicz, Principles of Fluorescence Spectroscopy, 3rd edition (Springer, 2006); Hermanson, Bioconjugate Techniques, Second Edition (Academic Press, 2008); and the like. Relevant parts of the above references are hereby incorporated by reference.

In some embodiments, the invention comprises optically based specific binding assays, such as immunoassays, in which optically labeled assay products, e.g. capture complexes, are identified using a subwavelength hole array illuminated by one or more excitation beams having wavelengths greater that the hole diameters and appropriate for the optical labels (that is, the excitation beams are capable of causing the optical labels to generate optical signals). As described more fully below, subwavelength hole arrays are well-known devices, e.g. Genet et al, Nature, 445: 39-46 (2007); Garcia-Vidal et al, Reviews of Modern Physics, 82: 729-787 (2010); and the like. For use with methods of the invention, hole diameters, geometric pattern, center-to-center distances, number of holes in an array, hole shape, depth, and like parameters are selected so that there is little or no propagation of light through the array and that an evanescence field is present at the exits of holes whenever exposed to an excitation beam. In some embodiments, holes are circular and have diameters less than the wavelength of any excitation beam. In other embodiments, holes are circular and have diameters less than one half of the wavelength of any excitation beam. In some embodiments, hole diameters selected (for example, in a kit) depend on the excitation wavelengths of optical labels used in an assay. In some embodiments, hole diameters are in the range of from 10 to 300 nm; or in the range of from 10 to 250 nm; or in the range of from 20 to 300 nm; or in the range of 20 to 200 nm. In some embodiments, center-to-center distances between holes in an array are greater than the diffraction limit of the optical signal wavelength of any optical labels employed. In some embodiments, center-to-center distances between holes in an array are at least twice such diffraction limit. In some embodiments, center-to-center distances between holes in an array are greater than the diffraction limit of the fluorescent emission maximum of any fluorescent labels employed. In some embodiments, center-to-center distances between holes in an array are in the range of from 300 nm to 20 μm; or from 500 nm to 20 μm; or from 750 nm to 1000 nm. In some embodiments, the number of holes in an array is greater than 4; or greater than 7; or greater than 9; or greater than 100. In some embodiments, the number of holes in an array is in the range of from 1 to 250000; or from 4 to 250000; or from 1 to 500000; or from 4 to 500000; or from 1 to 10000; or from 4 to 10000; or from 4 to 10000; or from 7 to 10000; or from 9 to 10000; or from 10 to 2000000; or from 10 to 1000000; or from 100 to 1000000. In some embodiments, holes of an array are arranged in a rectilinear pattern or in a triangular pattern. In some embodiments, assays of the invention employ one or more fluorescent labels. In some embodiments, assays of the invention employ from 1 to 16 fluorescent labels; or from 1 to 8 fluorescent labels; or from 1 to 6 fluorescent labels; or from 1 to 4 fluorescent labels.

In some embodiments, capture complexes comprise capture agents to which analytes and detection reagents are capable of binding directly or indirectly. In some embodiments, in the presence of a target analyte, a capture complex may comprise one or more capture agents, zero or more target analytes, and zero or more detection agents. And in the absence of a target analyte, a capture complex comprises one or more capture agents and zero detection agents. In some embodiments, each capture complex comprises a bead (or equivalently, a particle) which can pass through holes of a hole array. In some embodiments, beads have diameters less than 200 nm, or less than 100 nm, or less than 70 nm. In other embodiments, beads have diameters in the range of from 20 to 250 nm, or from 30 to 200 nm. In some embodiments, beads are magnetic beads which permit separation of captured analytes from a sample by the application of a magnetic field. The magnetic beads may be of different kinds, but are normally of matrix or core-shell type. The matrix type magnetic nanoparticle may comprise a porous silica, latex or polymer matrix filled with nanometer-sized magnetic particles. The core-shell type may comprise a core of nanometer-sized magnetic particles, covered with a non-magnetic coating that can be either silica, latex or a polymer. Nanometer-sized in this connection means smaller than 100 nm, preferably smaller than 50 nm but preferably larger than 10 nm. Said nanoparticle may comprise (γ-Fe2O3) or magnetite (Fe3O4). Guidance in using and making magnetic beads may be found in the following references incorporated herein by reference: Chuah et al, Nature Comm., 10: 2109 (2019); U.S. Pat. Nos. 7,396,589; 7,459,145; 7,547,473; 8,323,618; 11,305,351; and the like. In some embodiments, a capture agent comprises a magnetic bead having a size as described above, wherein such magnetic bead comprises one or more antibodies or antibody binding compositions attached.

In some embodiments, immunoassays of the invention are digital bead assays, e.g. Duffy (2023, cited above); Zhang et al, Anal. Chem., 89: 92-101 (2017); Bazu, SLAS Technology, 22(4): pt. 1, 369-386, and pt. 2, 387-405 (2017); and the like. Briefly, in a digital bead assay conditions during measurement comprise each bead bound to none or a single-digit integral number of analytes, wherein measurement occurs in the small volumes of the evanescence fields associated with each nanopore. In some embodiments, most beads during measurement comprise 0, 1 or 2 bound analytes. In other embodiments, ninety percent or more beads during measurement comprise 0, 1 or 2 bound analytes.

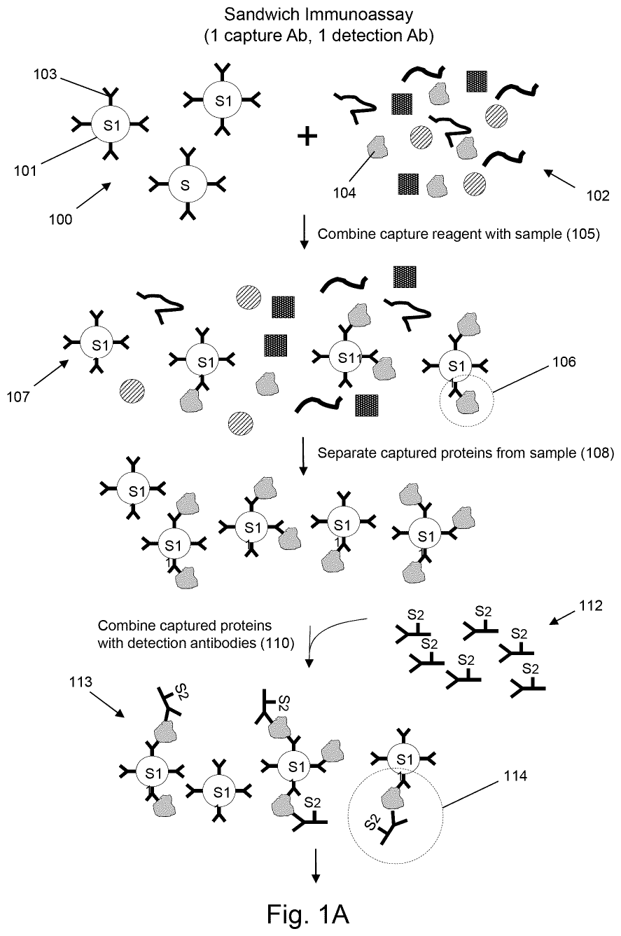

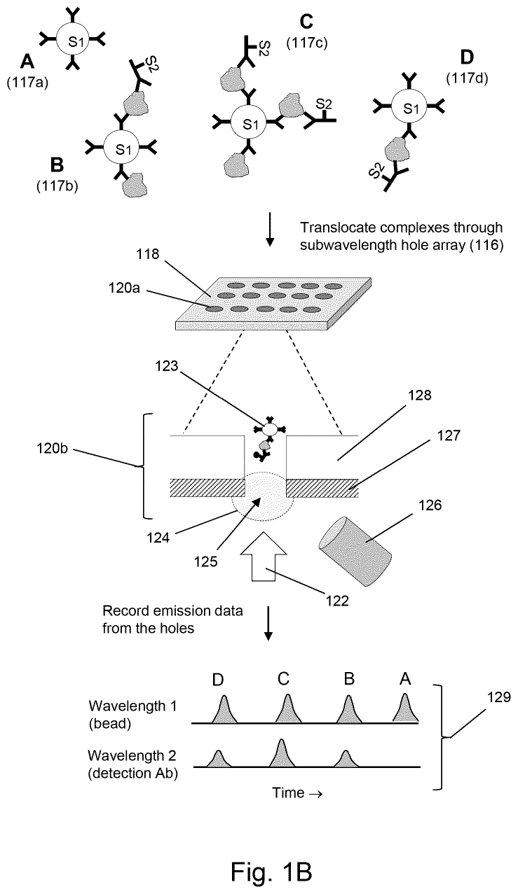

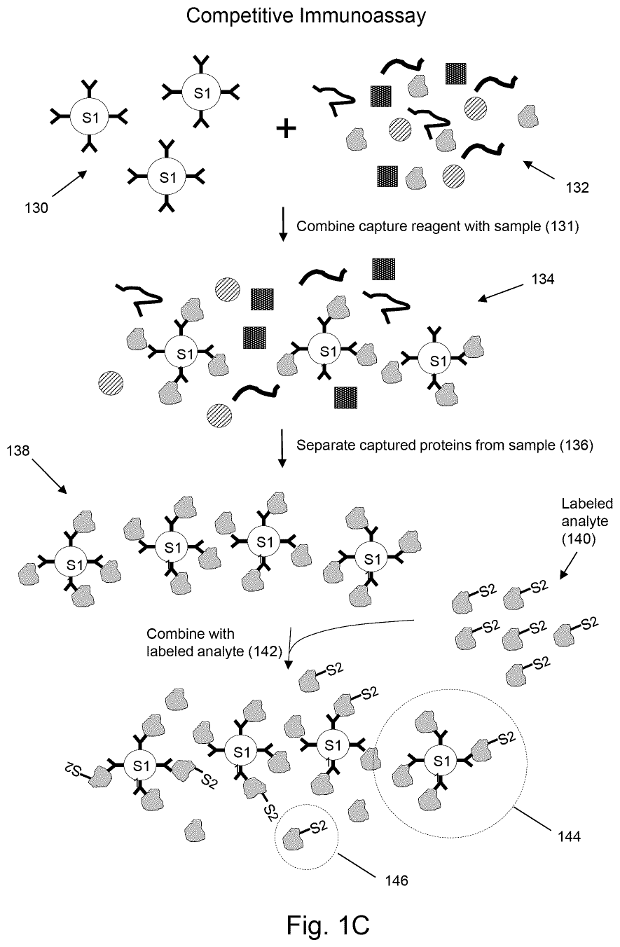

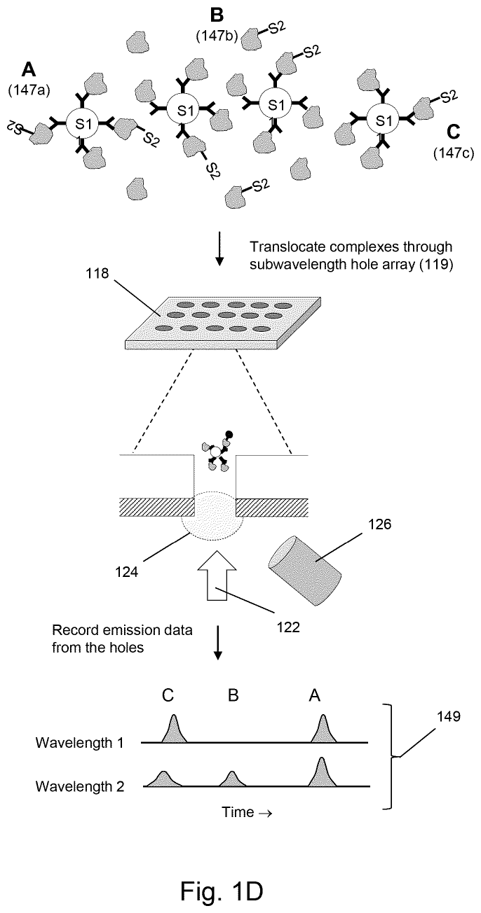

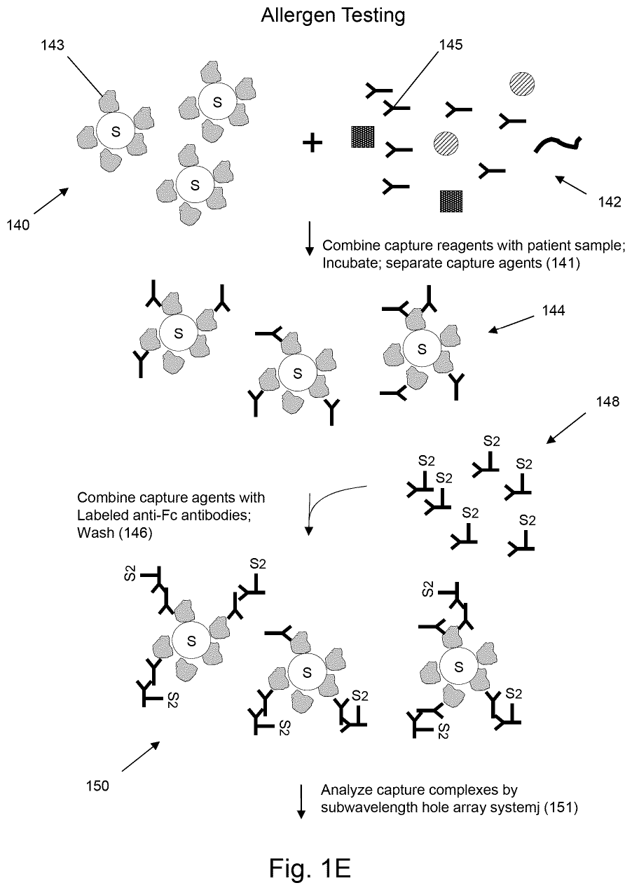

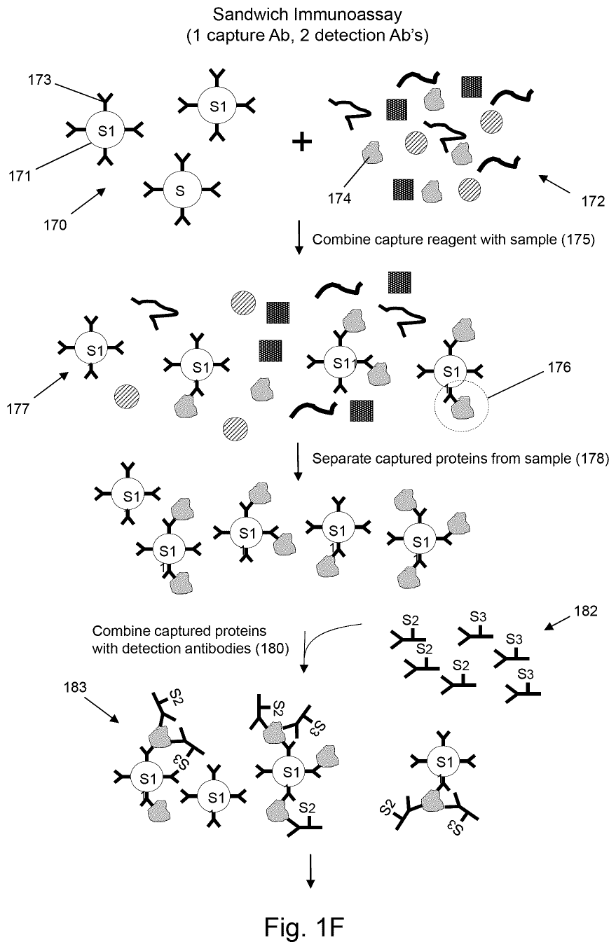

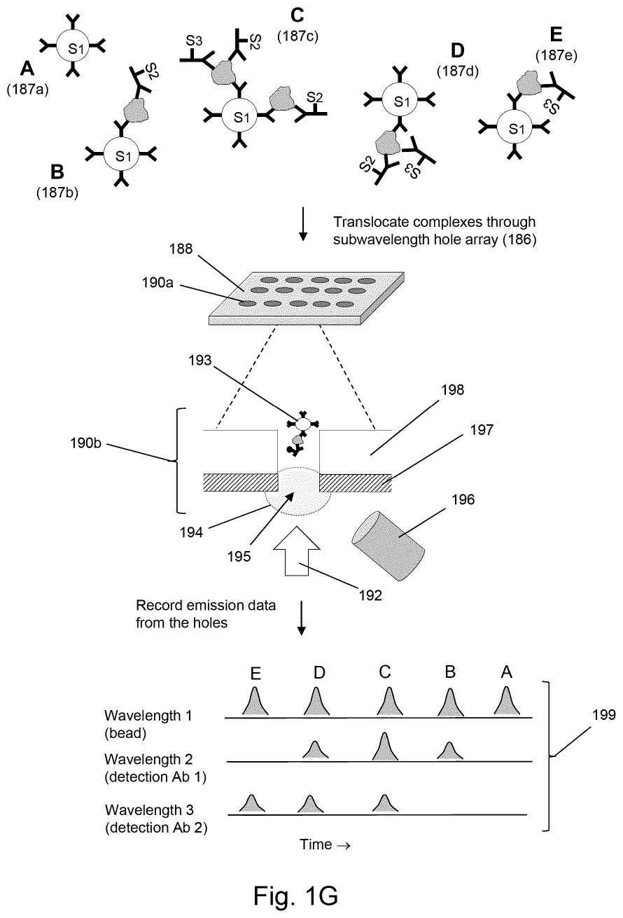

FIGS. 1A-1B, 1C-1D and 1E diagrammatically illustrate sandwich, competitive and allergen immunoassay embodiments. As used herein, the terms “immunoassay” and “antibody” are intended to include the use of other binding compounds, such as, antibody fragments, aptamers, and the like. In FIG. 1A, capture agents (100) comprising magnetic beads (101) with first optical label “S” and antibody (103) are combined with sample (102) containing a variety of objects in addition to target molecule (104). After incubation under conditions (105) for specific capture of target molecule (104) by antibody (103), capture agents-target molecule complexes are formed which comprise no (e.g. 107) or one or more target molecules (e.g. 106). Such complexes may be separated (108) from the sample by applying a magnetic field to attract the magnetic beads followed by washing. The captured target molecules are then combined (110) with detection antibodies (112) which are specific for a separate binding site on the target molecule to form capture complexes (113). For example, a separate binding site may be a separate epitope whenever the target molecule is a protein. As noted in FIG. 1A, capture complexes (113) comprise an optical label associated with particle (101) and one or more optical labels associate with detection antibodies (114). After washing to remove unbound detection antibody (112), capture complexes (113) are translocated through hole array (118) where they pass through an evanescence field of signal generation region (124) produced by incident excitation beam (122). Hole (120a) is shown in blown-up cross section (120b). Array (118) comprises support layer (128) which is usually a non-conductive material, such as, silicon nitride, silicon oxide, aluminum oxide, glass, or the like, and opaque layer (127) which is usually a conductive material, such as, gold, silver, aluminum, or the like. Signal generation region (124) (whose extent in the figure is only illustrative) is at exit (125) of the hole illustrated. Excitation beam (122) may be white light or it may comprise one or more restricted ranges of wavelengths, for example, comprising one or more laser excitation beams. In some embodiments, a signal generation region is radially symmetric with respect to the longitudinal axis of a hole of the hole array. In some embodiments, the intensity of the evanescence field in a signal generation region declines monotonically from a value at the opening (on the illuminated side) of a hole to the non-illuminated side of the array. In some embodiments, the intensity of the evanescence field has a half maximum value within 20 nm of the opening (on the illuminated side) of a hole. Detector (126) collects signals from optical labels of capture complexes (123) and transmits representations thereof to recording and/or processing apparatus. In embodiments where at least two distinct optical labels are employed, for example, one labeling the capture agent and one labeling the detection agent, optical signal of two different wavelengths are collected and recorded, as shown by illustrative data (129) for a capture agent without analyte, A (117a), and the three different capture complexes B, C and D (117b, c, d, respectively) at the top of the figure. Since in this embodiment all capture agents and complexes comprise a single bead (whose label may be inherent to the bead, e.g. magnetic luminescent nanoparticles, or attached) on average it will produce a signal of the same magnitude, as shown for wavelength 1). In embodiments in which a capture agent comprises antibodies for capturing a target molecule, one or more target molecules may be captured and one or more detection agents (also antibodies) may bind to target molecules to form a capture complex, e.g. in a sandwich configuration. Thus, the intensity of a signal collected from a second optical label may correspond to one or more optical labels of one or more detection agents, such as illustrated by capture complexes B and D that each have a single detection agent attached and capture complex C that has two detection agents attached. The different respective signals are reflected in the illustrative data in the wavelength 2 channel. In some embodiments, detection agents are antibodies or antibody binding compositions comprising a direct or indirect optical label. For example, a detection agent may comprise a cascade of two or more antibodies wherein (for example) a first antibody binds to a target analyte and a second antibody comprising an optical label binds to the first antibody or a hapten or Fc portion thereof. In some embodiments, such assays of the invention are capable of detecting a protein analyte at a concentration in a sample of at least 20 ng/mL; or at least 1 ng/mL; or at least 0.1 ng/mL.

FIGS. 1C and 1D illustrate the basic steps of a competitive immunoassay which may be especially useful where target molecules are small molecules, such as drugs, so that two or more binding sites are not present. As above, capture agents (130) are combined (131) with a sample (132) suspected of containing target molecules under conditions (134) that favor the capture of target molecules by capture agents. After incubation under such conditions, capture agents with captured target molecules (138) are separated (136) from the reaction mixture, after which they are combined (142) with a known amount of labeled target molecule (or analyte) (140) which then competes with unlabeled analyte for binding to capture agent, e.g. (144). Molecules (including unbound labeled analytes, e.g. (146)) and complexes of the resulting mixture may then be directly analyzed by translocation through a hole array or capture complexes may be separated from the resulting mixture and analyzed by translocation through a hole array. FIG. 1D illustrates the former type of analysis, wherein the resulting mixture includes at least three different labeled entities that generate optical signals as they pass through signal generation regions of holes of an array: A (147a) single bead, two labeled analytes; B (147b) single labeled analyte; and C (147c) single bead, one labeled analyte. After translocation through holes of hole array (118), the following illustrative data (149) is obtained: in the wavelength 1 channel a constant amplitude of signal is obtained for each bead passing through a hole (A and C) whereas no signal is obtained whenever a labeled analyte alone passes through a hole (B); in the wavelength 2 channel amplitude of a signal depends on whether a labeled analyte passes alone or bound to a capture agent in one or more copies. In some embodiments, such assays of the invention are capable of detecting a protein analyte at a concentration in a sample of at least 20 ng/mL; or at least 1 ng/mL; or at least 0.1 ng/mL.

FIG. 1E illustrates an assay for testing a patient's reaction to allergens. In this embodiment, capture agents (140) coated with allergen (143) are combined (141) with a patient sample (142), such as a blood sample, containing antibodies (145) specific for a given allergen(s). After incubation, capture agents comprising patient antibodies bound to the allergens are separated from the rest of the sample and combined (146) labeled anti-Fc antibodies (148) to form capture complexes (150). Capture complexes (150) are then analyzed (151) by translocating through a subwavelength hole array as described above.

FIGS. 1F-1G illustrate a sandwich assay with two detection antibodies for high sensitivity detection of a target analyte. In FIG. 1F, capture agents (170) comprising magnetic beads (171) with first optical label “S1” and antibody (173) are combined with sample (172) containing a variety of objects in addition to target molecule (174). After incubation under conditions (175) for specific capture of target molecule (174) by antibody (173), capture agents-target molecule complexes are formed which comprise no (e.g. 177) or one or more target molecules (e.g. 176). Such complexes may be separated (178) from the sample by applying a magnetic field to attract the magnetic beads followed by washing. The captured target molecules are then combined (180) with detection antibodies (182) which are specific for separate binding sites on the target molecule to form capture complexes (183) which comprise antibodies specific for a second epitope and comprising label S2 and antibodies specific for a third epitope and comprising label S3. As noted in FIG. 1F, capture complexes (183) comprise an optical label, S1, associated with particle (171) and one or more optical labels, e.g. S2 and S3, associate with detection antibodies (182). After washing to remove unbound detection antibodies (182), capture complexes (183) are translocated through hole array (188) where they pass through an evanescence field of signal generation region (194) produced by incident excitation beam (192). Hole (190a) is shown in blown-up cross section (190b). Array (188) comprises support layer (198) which is usually a non-conductive material, such as, silicon nitride, silicon oxide, aluminum oxide, glass, or the like, and opaque layer (197) which is usually a conductive material, such as, gold, silver, aluminum, or the like. Signal generation region (194) (whose extent in the figure is only illustrative) is at exit (195) of the hole illustrated. Excitation beam (192) may be white light or it may comprise one or more restricted ranges of wavelengths, for example, comprising one or more laser excitation beams. Detector (196) collects signals from optical labels of capture complexes (193) and transmits representations thereof to recording and/or processing apparatus. In embodiments where at least three distinct optical labels are employed, for example, one labeling the capture agent and two labeling the detection agents, optical signal of three different wavelengths are collected and recorded, as shown by illustrative data (199) for a capture agent without analyte, A (187a), and the three different capture complexes B, C and D (187b, c, d, e, respectively) at the top of the figure. Since all capture agents and complexes comprise a single bead (whose label may be inherent to the bead, e.g. magnetic luminescent nanoparticles, or attached) on average it will produce a signal of the same magnitude, as shown for wavelength 1). In embodiments in which a capture agent comprises antibodies for capturing a target molecule, one or more target molecules may be captured and one or more detection agents (also antibodies) may bind to target molecules to form a capture complex, e.g. in a sandwich configuration. Thus, the intensity of a signal collected from a second and third optical label may correspond to one or more optical labels of one or more detection agents, such as illustrated by capture complexes B and E that each have a single detection agent attached and capture complex C that has three detection agents attached. The different respective signals are reflected in the illustrative data in the wavelength 2 channel and wavelength 3 channel. In some embodiments, such assays of the invention are capable of detecting a protein analyte at a concentration in a sample of at least 20 ng/mL; or at least 1 ng/mL; or at least 0.1 ng/mL.

### Optical Labels

A wide variety of optical labels may be used with the invention. Optical labels may include virtually any moiety whose size is consistent with the nanopore limitations of the invention and that is capable of generating an optical signal in a non-propagating evanescence field of appropriate wavelength, such as may be produced in a zero mode waveguide, e.g. Levene et al, Science, 299: 682-684 (2003); U.S. Pat. No. 7,181,122, and the like. In some embodiments, optical labels comprise fluorescent labels. Optical labels may also comprise methods and compositions for signal amplification. In some embodiments, fluorescent labels comprise well-known fluorescent organic molecules including, but not limited to, rhodamine dyes, fluorescein dyes, cyanine dyes, fluorescent proteins, Alexa Fluor dyes, BODIPY dyes, quantum dots, and the like. Such dyes and compounds may be attached to antibodies and antibody binding compositions using conventional techniques, e.g. described in Hermanson (cited above), and like references. In some embodiments, optical labels comprise signal amplification components, such as tyramide signal amplification, rolling circle amplification (RCA), or the like, e.g. as disclosed in the following references: Akama et al, Anal. Chem., 88(14): 7123-7129 (2016); Konry et al, Anal. Chem., 81(14): 5777-5782 (2009); Schweitzer et al, Proc. Natl. Acad. Sci., 97(18): 10113-10119 (2000); Schweitzer et al, U.S. Pat. No. 6,531,283; Lizardi et al, U.S. Pat. Nos. 6,344,329; 6,183,960; and 5,854,033; and the like, which are incorporated herein by reference. In some embodiments, optical labels comprise RCA signal amplification. In some embodiments, RCA amplicons are labeled with fluorescently labeled oligonucleotides, e.g. as disclosed in Schweitzer (2000, cited above). In some embodiments, such fluorescently labeled oligonucleotides comprise molecular beacon probes, e.g. Tyagi et al, U.S. Pat. No. 5,925,517, which is incorporated herein by reference.

### Instrumentation

In one aspect, the invention includes systems comprising a hole array and capture complexes which, in turn, comprise beads or particles, analytes and detection agents. In some embodiments, systems of the invention comprise one or more light sources for generating excitation beams for illuminating optical labels of capture complexes. In some embodiments, systems of the invention further comprise one or more detectors to detect optical signals generated by the optical labels, e.g. as described in Sawafta et al, Nanoscale, 6(12): 6991-6996 (2014); Soni et al, U.S. patent publication US2012/0135410; and like references, which are incorporated herein by reference. In some embodiments, systems comprise a cis reservoir (or chamber) on the non-illuminated side of a hole array and a trans reservoir (or chamber) on the illuminated side of the hole array, wherein the cis reservoir and the trans reservoir are in fluid communication with each other through the holes of the hole array. In some embodiments, the systems are configured to translocate capture complexes in the cis reservoir to the trans reservoir, such that such translocated capture complexes traverse signal generation regions of the holes. In some embodiments, capture complexes are electrically charged and are translocated through the holes by applying an electrical field. In some embodiments, capture complexes comprise magnetic beads and are translocated through the holes by applying a magnetic field. In some embodiments, capture complexes are translocated through the holes by generating a positive pressure gradient between the cis reservoir and the trans reservoir.

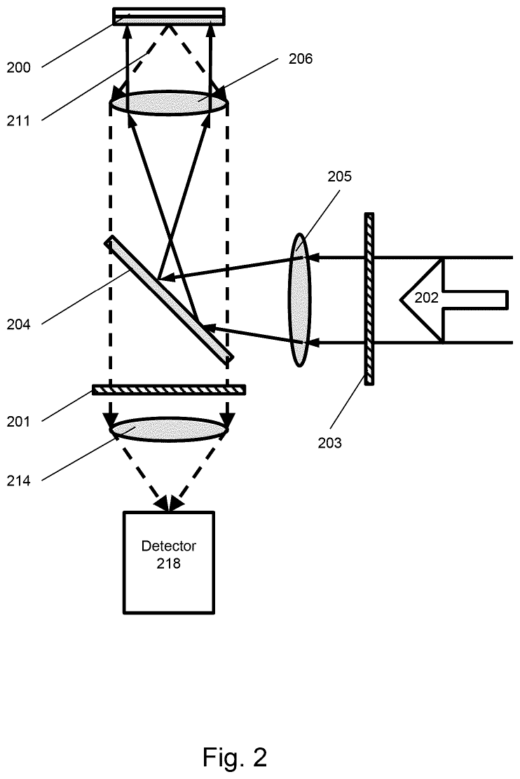

In some embodiments, an epi-illumination system, in which excitation beam delivery and optical signal collection occur through a single objective, may be used for direct illumination of optical labels on capture complexes and other labeled components. The basic components of an epi-illumination system for use with the invention are illustrated in FIG. 2. Excitation beam (202) is filtered through an excitation filter (203) and then focused by a lens (205). The converged beam is directed towards a dichroic mirror (204), which reflects it through the objective lens (206) onto a layered membrane (200), in which labels are excited directly to emit an optical signal, such as a fluorescent signal, or are excited indirectly via a FRET interaction to emit an optical signal. Such optical signal (211) is collected by the same objective lens (206) and directed to the dichroic mirror (204), which is selected so that it transmits the light of optical signals (211) but reflects the light of the excitation beam (202). Transmitted optical signals (211) pass through an emission filter (201) and a lens (214), which focuses it onto a detector (218).

The present invention is directed to methods and devices for optically-based analysis of molecules, such as proteins, which comprise subwavelength hole arrays with one or more light-blocking layers, that is, one or more opaque layers. Typically subwavelength hole arrays are fabricated in thin sheets of material, such as, silicon, silicon nitride, silicon oxide, aluminum oxide, or the like, which readily transmit light, particularly at the thicknesses used, e.g. less than 50-100 nm. In one aspect, the invention addresses this problem by providing subwavelength hole arrays with one or more light-blocking layers that reflect and/or absorb light from an excitation beam, thereby reducing background noise for optical signals generated at intended sites associated with subwavelength holes of an array. In some embodiments, this permits optical labels in intended reaction sites (such as detection zones or signal generation zones described more fully below) to be excited by direct illumination. In some embodiments, an opaque layer may be a metal layer. Such metal layer may comprise Pt, Pd, Cr, Sn, Al, V, Ti, Ni, Mo, Ta, W, Au, Ag or Cu. In some embodiments such metal layer may comprise Al, Au, Ag or Cu. In still other embodiments, such metal layer may comprise aluminum or gold, or may comprise solely aluminum. The thickness of an opaque layer may vary widely and depends on the physical and chemical properties of material composing the layer. In some embodiments, the thickness of an opaque layer may be at least 5 nm, or at least 10 nm, or at least 40 nm. In other embodiments, the thickness of an opaque layer may be in the range of from 5-300 nm; or in the range of from 5-100 nm; or in the range of from 10-80 nm. An opaque layer need not block (i.e. reflect or absorb) 100 percent of the light from an excitation beam. In some embodiments, an opaque layer may block at least 10 percent of incident light from an excitation beam; in other embodiments, an opaque layer may block at least 50 percent of incident light from an excitation beam.

In some embodiments, whenever the opaque coating or layer is a metal, a nearest-neighbor hole distance and excitation beam wavelength are selected to minimize plasmon-mediated extraordinary transmission through the hole array. Guidance for such selections are disclosed in the following references that are incorporated by reference: Ebbesen et al, Nature, 391: 667-669 (1998); Ebbesen et al, U.S. Pat. Nos. 5,973,316; 6,040,936; 6,236,033; 6,856,715; 7,057,151; 7,248,756; 8,174,696; Gur et al, Optics Comm., 284: 3509-3517 (2011); Ghaemi et al, Physical Review B, 58: 6779-6782 (1998); Pacifici et al, Optics Express, 16(12): 9222-9238 (2008); and the like. In some embodiments, a nearest-neighbor hole distance (or expected nearest-neighbor hole distance, for example, in a random (e.g. Poisson distributed) array of holes) is selected which approximately equals an excitation wavelength, for example, for exciting optical labels.

In some embodiments, the invention comprises hole arrays with one or more light-blocking layers, that is, one or more opaque layers. Typically hole arrays are fabricated in thin sheets of material, such as, silicon, silicon nitride, silicon oxide, aluminum oxide, or the like, which readily transmit light, particularly at the thicknesses used, e.g. less than 50-100 nm. For electrical detection of analytes this is not a problem. However, in optically-based detection of labeled molecules translocating holes, light transmitted through an array invariably excites materials outside of intended reaction sites, thus generates optical noise, for example, from nonspecific background fluorescence, fluorescence from labels of molecules that have not yet entered a hole, or photoluminescence from the membrane itself, or the like. In one aspect, the invention addresses this problem by providing hole arrays with one or more light-blocking layers that reflect and/or absorb light from an excitation beam, thereby reducing background noise for optical signals generated at intended reaction sites associated with holes of an array. In some embodiments, an opaque layer may be a metal layer. Such metal layer may comprise Sn, Al, V, Ti, Ni, Mo, Ta, W, Au, Ag or Cu. In some embodiments such metal layer may comprise Al, Au, Ag or Cu. In still other embodiments, such metal layer may comprise aluminum or gold, or may comprise solely aluminum. The thickness of an opaque layer may vary widely and depends on the physical and chemical properties of material composing the layer. In some embodiments, the thickness of an opaque layer may be at least 5 nm, or at least 10 nm, or at least 40 nm. In other embodiments, the thickness of an opaque layer may be in the range of from 5-100 nm; in other embodiments, the thickness of an opaque layer may be in the range of from 10-80 nm. An opaque layer need not block (i.e. reflect or absorb) 100 percent of the light from an excitation beam. In some embodiments, an opaque layer may block at least 10 percent of incident light from an excitation beam; in other embodiments, an opaque layer may block at least 50 percent of incident light from an excitation beam.

Holes in opaque layers or coatings may be fabricated on solid state membranes by a variety of techniques known in the art including, not limited to, lift-off, focused ion-beam milling, wet or dry etching, and the like. Material deposition techniques may be used including chemical vapor deposition, electrodeposition, epitaxy, thermal oxidation, physical vapor deposition, including evaporation and sputtering, casting, and the like. In some embodiments, atomic layer deposition may be used, e.g. U.S. Pat. No. 6,464,842; Wei et al, Small, 6(13): 1406-1414 (2010), which are incorporated by reference.

A synthetic hole array, or solid-state hole array, may be created in various forms of solid substrates, examples of which include but are not limited to silicones (e.g. Si₃N₄, SiO₂), metals, metal oxides (e.g. Al₂O₃) plastics, glass, semiconductor material, and combinations thereof.

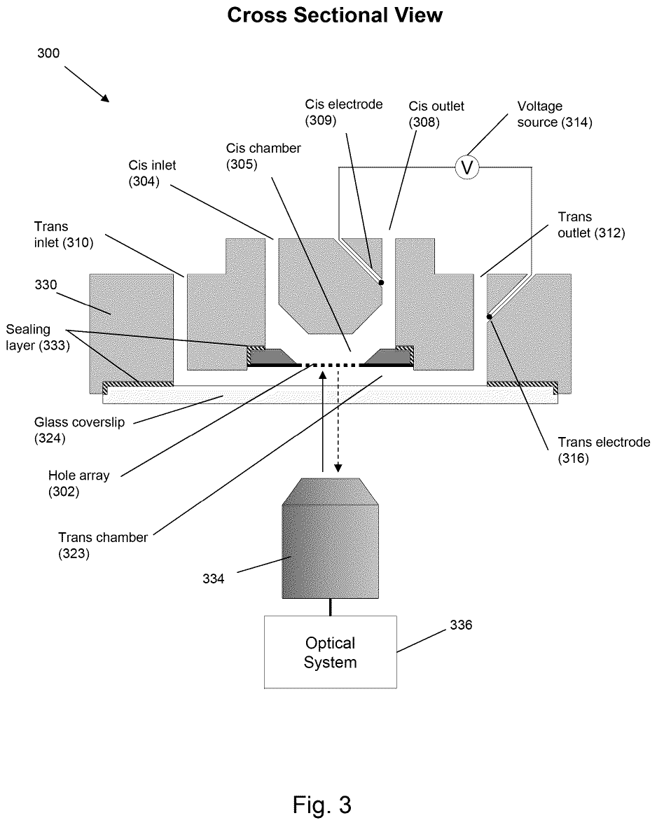

For use in an assay, hole arrays may be mounted in a microfluidic device which provides inlets, outlets, chambers, channels and like features for delivering capture complexes to hole arrays. FIG. 3 illustrates a design of one embodiment of such a microfluidic device. In this embodiment, sample preparation, combination with capture agents and detection agents and separation of capture complexes and/or other labeled components are performed using other conventional apparatus. Such compounds for measurement and analysis are referred to herein as “capture complexes,” however, such compounds may include other ingredients, such as, labeled standards, diluents, viscosity modifiers, or the like. FIG. 3 shows a cross sectional view of cartridge or microfluidic device (300) so that microfluidic features may be seen. Hole array (302) is disposed between cis chamber (305) and trans chamber (323) below hole array (302). The terms “cis” and “trans” in respect to chambers (305) and (323) are conventional terms that indicated the direction of translocation of a charged molecule under the influence of an electrical field or voltage gradient produced by a voltage source, e.g. (314). In some embodiments, microfluidics device (300) may be configured to translocate a negatively charged molecule or complex from cis chamber (305) through hole array (302) to trans chamber (323). In other embodiments, microfluidics device (300) may be configured to translocate a positively charged molecule or complex from cis chamber (305) through hole array (302) to trans chamber (323). In some embodiments, forces other than electrical forces, e.g. pressure differential, magnetic force, or the like, may be used to translocate capture complexes through hole array (302). Capture complexes in a carrier fluid or buffer are loaded via cis inlet (304) to cis chamber (305) and to the top of hole array (302). Displaced fluid from chamber (305) may exit through cis outlet (308). Cis inlet (304), cis chamber (305), cis outlet (308), trans inlet (310), trans chamber (323), trans outlet (312), and other features may be fabricated in conventional materials, e.g. silicon, glass, plastic, or the like, to produce layer (330). Coverslip (324) and hole array (302) are attached to layer (330) by sealing layer (333). Electrodes (309 and 316) from voltage source (314) are contacted with the cis chamber by port (309) and with the trans chamber by port (316), respectively. In this embodiment, trans chamber inlet (310) and outlet (312) are located on the top of microfluidics device (300) to avoid interference with placement of microscope objective (334) immediately below hole array (302) for illumination and signal collection. In operation, excitation light is transmitted through objective (334) to hole array (302) and optical signals from capture complexes are collected by the same objective using, for example, an optical system (336) described in FIG. 2.

### Definitions

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immuology, 6^{th} edition (Saunders, 2007); Murphy, Janeway's Immunobiology, 8^{th} edition (Garland Science).

“Analyte” means a substance, compound, or component in a sample whose presence or absence is to be detected or whose quantity is to be measured. Analytes include but are not limited to peptides, proteins, polynucleotides, polypeptides, oligonucleotides, organic molecules, haptens, epitopes, parts of biological cells, posttranslational modifications of proteins, receptors, complex sugars, vitamins, hormones, microorganisms, bacteria, viruses, and the like. There may be more than one analyte associated with a single molecular entity, e.g. different phosphorylation sites on the same protein. In some embodiments, analytes are proteins that are biomarkers for conditions of health or disease. In some embodiments, analytes comprise one or more biomarker proteins for a cancer,

“Antibody” means an immunoglobulin that specifically binds to, and is thereby defined as complementary with, a particular spatial and polar organization of another molecule. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular polypeptide is maintained. Guidance in the production and selection of antibodies for use in immunoassays, including such assays employing releasable molecular tag (as described below) can be found in readily available texts and manuals, e.g. Harlow and Lane, Antibodies: A Laboratory Manual (Cold Spring Harbor Laboratory Press, New York, 1988); Howard and Bethell, Basic Methods in Antibody Production and Characterization (CRC Press, 2001); Wild, editor, The Immunoassay Handbook (Stockton Press, New York, 1994), and the like. The term “antibody” as used herein also includes aptamers.

“Antibody binding composition” or “antibody binding compound” means a molecule or a complex of molecules that comprises one or more antibodies, or fragments thereof, and derives its binding specificity from such antibody or antibody fragment. Antibody binding compositions include, but are not limited to, (i) antibody pairs in which a first antibody binds specifically to a target molecule and a second antibody binds specifically to a constant region of the first antibody; a biotinylated antibody that binds specifically to a target molecule and a streptavidin protein, which protein is derivatized with moieties such as molecular tags or photosensitizers, or the like, via a biotin moiety; (ii) antibodies specific for a target molecule and conjugated to a polymer, such as dextran, which, in turn, is derivatized with moieties such as molecular tags or photosensitizers, either directly by covalent bonds or indirectly via streptavidin-biotin linkages; (iii) antibodies specific for a target molecule and conjugated to a bead, or microbead, or other solid phase support, which, in turn, is derivatized either directly or indirectly with moieties such as molecular tags or photosensitizers, or polymers containing the latter.

“Complex” as used herein means an assemblage or aggregate of molecules in direct or indirect contact with one another. In one aspect, “contact,” or more particularly, “direct contact” in reference to a complex of molecules, or in reference to specificity or specific binding, means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules is stable in that under assay conditions the complex is thermodynamically more favorable than a non-aggregated, or non-complexed, state of its component molecules.

“Epitope” or “antigenic determinant” means any molecule that may be recognized in a specific manner by an antibody or a derivative thereof. In some embodiments, an epitope is a portion of a protein. Epitopes may include posttranslational modifications, such as carbohydrate or lipid moieties. In some embodiments, epitopes may be peptides, polysaccharides, or lipids, small molecules (e.g. <900 MW) or combinations thereof.

“Kit” means any delivery system for delivering materials or reagents for carrying out a method of the invention. In the context of reaction assays, such delivery systems include systems that allow for the storage, transport, or delivery of reaction reagents (e.g., capture agents, detection agents, etc. in the appropriate containers) and/or supporting materials (e.g., buffers, written instructions for performing the assay etc.) from one location to another. For example, kits may include one or more enclosures (e.g., boxes) containing the relevant reaction reagents and/or supporting materials. Such contents may be delivered to the intended recipient together or separately. For example, a first container may contain a capture agent for use in an assay, while second and third containers may contain detection agents and a cartridge containing a subwavelength hole array.

“Protein” refers to a polypeptide, usually synthesized by a biological cell, folded into a defined three-dimensional structure. Proteins are generally from about 5,000 to about 5,000,000 or more in molecular weight, more usually from about 5,000 to about 1,000,000 molecular weight, and may include posttranslational modifications, such acetylation, acylation, phosphorylation, ubiquitination, or the like, e.g. Wold, F., Post-translational Protein Modifications: Perspectives and Prospects, pgs. 1-12 in Post-translational Covalent Modification of Proteins, B. C. Johnson, Ed., Academic Press, New York, 1983. Proteins include, by way of illustration and not limitation, cytokines or interleukins, enzymes such as, e.g., kinases, proteases, galactosidases and so forth, protamines, histones, albumins, immunoglobulins, scleroproteins, phosphoproteins, mucoproteins, chromoproteins, lipoproteins, nucleoproteins, glycoproteins, T-cell receptors, proteoglycans, somatotropin, prolactin, insulin, pepsin, proteins found in human plasma, blood clotting factors, blood typing factors, protein hormones, cancer antigens, tissue specific antigens, peptide hormones, nutritional markers, tissue specific antigens, synthetic peptides, and the like.

The term “sample” in the present specification and claims is used in a broad sense. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention. In some embodiments, a sample comprises serum.

“Specific” or “specificity” in reference to the binding of one molecule to another molecule, such as a binding compound, or probe, for a target analyte, means the recognition, contact, and formation of a stable complex between the probe and target, together with substantially less recognition, contact, or complex formation of the probe with other molecules. In one aspect, “specific” in reference to the binding of a first molecule to a second molecule means that to the extent the first molecule recognizes and forms a complex with another molecules in a reaction or sample, it forms the largest number of the complexes with the second molecule. In one aspect, this largest number is at least fifty percent of all such complexes form by the first molecule. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak noncovalent chemical interactions, such as Van der Waal forces, hydrogen bonding, ionic and hydrophobic interactions, and the like, dominate the interaction of the molecules. As used herein, “stable complex” in reference to two or more molecules means that such molecules form noncovalently linked aggregates, e.g. by specific binding, that under assay conditions are thermodynamically more favorable than a non-aggregated state.

What is claimed is:

1. A method of determining the presence and amounts of one or more analytes in a sample, the method comprising:
providing a hole array comprising a solid phase membrane and an opaque layer co-extensive therewith and a plurality of holes there through, wherein (i) the opaque layer substantially prevents excitation light from passing through the hole array, and (ii) each hole has an exit and a diameter less than a wavelength of excitation light such that a signal generation region is formed adjacent to its exit whenever excitation light is incident to the opaque layer;
combining a sample with capture agents and associated detection agents to form one or more capture complexes each having one or more optical labels, wherein each capture agent and associated detection agents is specific for an analyte;
directing excitation light to the opaque layer of the hole array to produce signal generation regions at the exits of the holes;
translocating capture complexes through the hole array from a side opposite the opaque layer so that the one or more optical labels of each capture complex produce one or more optical signals as the capture complex passes through a signal generation region at the exits of the holes;
detecting for each hole the one or more optical signals of the translocated capture complexes;
determining the presence or absence and the quantities of each of the one or more analytes in the sample from the optical signals collected from the capture complexes.

2. The method of claim 1 wherein said opaque layer is a metal layer.

3. The method of claim 2 wherein said metal layer comprises an aluminum layer or a gold layer.

4. The method of claim 2 wherein said directing and detecting are implemented with an epi-illumination system.

5. The method of claim 1 wherein said capture agent comprises a particle having a diameter less than said diameter of said holes.

6. The method of claim 5 wherein said particle is magnetic.

7. The method of claim 5 wherein said particle is charged.

8. The method of claim 1 wherein said optical labels are fluorescent labels.

9. The method of claim 1 wherein each of said capture complexes comprises an analyte sandwiched between a capture antibody and a detection antibody.

10. The method of claim 9 wherein said one or more analytes comprise one or more proteins.

11. The method of claim 1 wherein each of said capture complexes comprises a capture antibody and an unlabeled analyte or a labeled analyte.

12. The method of claim 1 wherein each of said capture agents comprise an allergen.

13. A method of determining the presence and amounts of one or more analytes in a sample, the method comprising:
combining a sample with capture agents and associated detection agents to form one or more capture complexes each having one or more optical labels, wherein each capture agent and associated detection agents is specific for an analyte;
translocating capture complexes through a hole array illuminated with excitation light, wherein the hole array comprises a solid phase membrane and an opaque layer co-extensive therewith and a plurality of holes there through, such that (i) the opaque layer substantially prevents excitation light from passing through the hole array, and (ii) each hole has an exit and a diameter less than a wavelength of excitation light such that a signal generation region is formed adjacent to its exit;
detecting for each hole the one or more optical signals of the translocated capture complexes; and
determining the presence or absence and the quantities of each of the one or more analytes in the sample from the optical signals collected from the capture complexes.

14. The method of claim 13 wherein said opaque layer is a metal layer.

15. The method of claim 13 wherein each of said capture agent comprises a particle having a diameter less than said diameter of said holes.

16. The method of claim 15 wherein ninety percent or more of said one or more optical signals indicate said particles comprise 0, 1 or 2 bound analytes

17. The method of claim 15 wherein said particle is magnetic.

18. The method of claim 15 wherein said particle is charged.

19. The method of claim 13 wherein said optical labels are fluorescent labels.

20. The method of claim 13 wherein each of said capture complexes comprises an analyte sandwiched between a capture antibody and a detection antibody.

21. The method of claim 20 wherein said one or more analytes comprise one or more proteins.

22. The method of claim 13 wherein each of said capture complexes comprises a capture antibody and an unlabeled analyte or a labeled analyte.

23. The method of claim 13 wherein each of said capture agents comprise an allergen.

24. A system for determining the presence and amounts of one or more analytes in a sample, the system comprising:
a hole array comprising a solid phase membrane and an opaque layer co-extensive therewith and a plurality of holes therethrough, such that (i) the opaque layer substantially prevents excitation light directed thereto from passing through the hole array, and (ii) each hole has an exit and a diameter less than a wavelength of excitation light such that a signal generation region is formed adjacent to its exit;
one or more beads each having a diameter less than the diameters of the holes of the hole array and each comprising a capture agent specific for an analyte, wherein the one or more beads form capture complexes in the presence of analytes and detection agents, such that the capture complexes generate one or more optical signals as such capture complexes translocate through the signal generation regions of the holes of the hole array, thereby generating optical signals which are indicative of the presence and amounts of the analytes in the sample.

25. The system of claim 24 further comprising;
One or more light sources that direct one or more excitation beams to said opaque layer of said hole array;
a cis reservoir on a side of said hole array opposite of said opaque layer; and
a trans reservoir on a side of said hole array the same as the excitation beam, wherein said capture complexes are translocated from the cis reservoir to the trans reservoir through said signal generation regions of said holes of said hole array.

26. The system of claim 25 further comprising one or more detectors for collecting said optical signals.
